(19) United States
(12) Patent Application Publication
(10) Pub. No.: US 2021/0031014 A1
(43) Pub. Date: Feb. 4, 2021

(54) Touch and Virtual Application for Art, Designs and Tattoos

(71) Applicant: Alyssa Lynn Chomeczko, Calgary (CA)

(72) Inventor: Alyssa Lynn Chomeczko, Calgary (CA)

(21) Appl. No.: 16/527,121

(22) Filed: Jul. 31, 2019

(51) Int. Cl. A61M 37/00; G06F 3/01; G06T 19/20; G06F 3/0481

(52) U.S. Cl. CPC A61M 37/0076; G06F 3/04815; G06T 19/20; G06F 3/017

(57) ABSTRACT

Disclosed is the system for virtual design including a computing device. The computing device includes a processor, a memory, and a displaying device. The memory stores instructions executable by the processor. The instructions can include a first application. The displaying device is coupled to the processor. In some embodiments, the processor runs a first application. The first application is adapted to assist designers with the virtual tattoo design. The first application is integrated with the computing device and includes a plurality of design features from which the designers choose to modify a design into a final design.

### REFERENCE TO PENDING APPLICATIONS

This application does not claim benefit of any issued U.S. patent or pending applications.

### BACKGROUND OF THE INVENTION

### Field of the Invention

The present invention relates generally to a virtual input device, and in particular to devices to virtually input art designs.

### Background

A tattoo artist creates a stencil of the tattoo design before he takes a needle to the skin. The artist draws the stencil with a special ink that transfers to the skin. This provides an outline of the tattoo design, which allows the artist to work quickly and accurately. Creating a tattoo stencil takes quite a bit of practice to get it right. Artists traditionally use a sheet of tracing paper, a fine-tipped marker, a dip pen and stencil fluid to sketch a design. However, regular modification during designing and until a final design is attained, makes the design task a time consuming and sometimes tedious task.

US20120307304A1 discloses an on-site creation of custom articles for receiving personalized artwork such as digitally-input freehand drawings and printing the artwork on-site and substantially immediately onto articles of apparel, merchandise, and the like. In certain aspects, the invention provides system for creating a customized article that includes an input device to receive data corresponding to a freehand drawing. The system stores the data in a memory and renders the data to create an image file for printing (e.g., in a raster or vector image format). The image file is printed, and the printed image is transferred to an article such as a garment, poster, mug, or mouse pad. In some respects, the invention provides systems and methods for creating a customized temporary tattoo in real-time. Methods of the invention include using a device that digital captures a freehand drawing to create the digital input. Suitable devices include electronic drawing tablets, touchscreens, computers such as laptops, desktops and tablet computers, projection screens, infrared drawing tools, cameras and other imaging devices. In certain embodiments, the digital input device comprises a rear-projection screen sensitive to infrared input. Methods includes using image manipulation software. Software can allow the customer to perform features such as undo, cut, copy, paste, edit color levels, brightness, contrast, invert, select portions, apply a filter, insert text, composite material from multiple files, sources, or input sessions, and perform other digital manipulation.

US20140310640A1 discloses an interactive digital art apparatus. The interactive digital art apparatus includes a display unit, an input interface unit, a posture tracking unit, and a processing unit. The display unit visualizes a virtual canvas. The input interface unit is manipulated by a user, and outputs input interface information attributable to an action in which the user draws a picture on the virtual canvas in front of the display unit. The posture tracking unit tracks a direction in which the face of the user is directed, and the posture of the input interface unit. The processing unit draws a picture on the virtual canvas based on the input interface information, the direction in which the face of the user is directed, and the posture of the input interface unit. Accordingly, the invention has been made keeping in mind the above problems occurring in the conventional art, and an object of the present invention is to provide an interactive digital art apparatus, which enables various artistic activities, such as making a painting, a drawing, a sketch, graffiti, etc. on a virtual canvas, to be performed, and also enables the virtual performance of a musical instrument to be made using a user interface. The interactive digital art may further include a Graphic User Interface (GUI) provision unit configured to include a plurality of menu options and to display the plurality of menu options on the virtual canvas in response to a request from the user. The plurality of menu options may include menu options for the replacement of paint, varnish, spray color and background color, and the storage, printing, e-mail transmission, mobile terminal multimedia transmission and website posting of results. The input interface unit may be implemented as various mockups having various shapes, such as brush, roller, stick and spray can shape. If the input interface unit is a mockup having a brush or roller shape, input interface information (for example, input interface parameters) may include information about pressing force, the mix of water, etc.

US20140245116A1 discloses a system and method for providing a digital art platform. Data structure(s) configured for storing information associated with digital art may be provided. A GUI operable on each of a plurality of client computing devices is provided and configured to provide a virtual canvas. A transformation module can be configured to receive at least one instruction and to modify the digital art when executed by at least one processor in response to the at least one instruction. Moreover, an update module can be included to transmit at least one instruction to at least one second of the plurality of computing devices. The modified digital art can be provided in each respective canvas of the at least one second of the plurality of computing devices in response to the at least one instruction. Users can focus on creative steps, such as applying paint or sound to an object, and the present application processes such steps to transform the object accordingly. In one or more implementations, processes happen automatically, and changes are saved, and thereafter stored in one or more servers that may be remotely located. In one or more implementations, processing occurs “locally” and within an internet web browser software application. Alternatively, processing occurs on a computing device that is remotely located from the user's computing device. In either instance, processing operations appear seamless to the user. user workstations may include, for example, mobile computing devices such as tablet computing devices, smartphones, personal digital assistants or the like. Further, printed output is provided, for example, via output printers. The application provides a quasi-physical behavior of paint. In one or more implementations, digital screen-printing or stenciling functionality is provided. An object (e.g., image file) that is added to a field can be converted into a stencil.

US20110276891A1 discloses computer-based methods, systems, and apparatus, including computer programs encoded on a computer storage medium, for presenting a first virtual art canvas at a graphical user interface; receiving instructions from a remote control unit to apply one or more markings to the virtual art canvas, where the one or more markings are applied according to the remote control unit's movements relative to a stationary sensor; and displaying one or more indicators on the virtual canvas to guide a user's movement of the remote control unit to create a virtual piece of art on the first virtual art canvas based, at least in part, on movements of the remote control unit. Certain embodiments include storing one or more virtual pieces of art in an electronic database; and enabling the user to access the electronic database over a network and to select among the stored virtual pieces of art one of the virtual pieces of art to create. In a virtual color palate that the user can access to select the color of virtual paint to apply to the canvas. In a typical implementation, the virtual color palate is accessible whether the user is creating art in guided-mode or in free-hand mode.

US20080036771A1 discloses a pen-based system allows users, such as artists, graphic designers and illustrators, and the like, to create accurate curve models by sketching. A tool set implemented in software that runs on a computer system combines paper sketch metaphors and a minimalist gesture set. This tool set provides an environment where the artists can focus on the creative task at hand rather than the details of operating software. A pen-based drawing method and system allows users to create and edit three dimensional (“3D”) objects by sketching. Pen input device may illustratively have a pen stylus and a sketch tablet on which a user can “draw” using pen. Pen input device captures the strokes drawn by a user on sketch tablet using pen and inputs them into computer. In an aspect, various perspective hints and engineering constraints are provided to help designers sketch easily. All the commands are classified in terms of frequency. For less frequent commands, such as changing 2D/3D and selecting a 3D curve creation method, the user pops up an on-screen menu and chooses a command. For infrequent commands, conventional menus are used.

U.S. Pat. No. 8,817,017B2 discloses method and system for 3D hand-painting and 3D hand-drawing. The system corresponding to the invented method comprises: an electronic monitor and computer, means for 3D digital vision, multi-axis input control device allowing digital painting or drawing on the canvas, additional input control device for virtual changing the position of the canvas between the painter and the monitor, means for 3D image presentation. The system and method provide painting or drawing on the electronic canvas for each of the virtual positions of the canvas by changing this position with the input control device and verifying it with the means for 3D digital vision. The system provides complete digital 2D images of the painting for the right and the left eyes, which are used for presentation of 3D painting by available means for 3D vision. The system comprises an electronic canvas (computer monitor, TV screen, projection screen, display of the mobile device, etc.) controlled by computer. A key component of the system is a hand-held electronic pen or brush allowing painting or drawing by hand on the canvas. Obviously, this brush/pen can be digital, wireless and provide many other functions useful for inputting information into digital processing systems. It might be useful to have this device controlled by the other than painting hand. Multiple control buttons/joysticks can be used for different fingers increasing the number of functions to be controlled. The control buttons/joysticks can be one-axis, two axis or three-axis additionally increasing the number of controlled functions. If thumb and four fingers are used and each of the buttons/joysticks is a three-axis device then control functions can be used simultaneously. For example, controlling functions of the digital painting process can be chosen from a group of: action, turning on and off, navigation of a cursor on the display of the electronic device, scrolling, zooming, shadowing, screening, selecting, deleting, restoring, saving, opening, closing, searching, setting up, previewing, undoing, clearing, repeating, pasting, finding, replacing, inserting, formatting, color selection, color mixing, line or stroke width, brush size, swatch size, sponge size, eraser size, the canvas virtual position, the depth of the focal plane around the canvas virtual position, special effects or combination.

US20130229390A1 discloses a graphics application for simulating natural media drawing and painting may model a tablet stylus as if it were a virtual projector, and as if a 2D brush tip image were projected on a virtual canvas. The application may compute a texture projection based on the values of configurable parameters of the application and/or 6 DOF data collected from the tablet stylus and/or the tablet. This texture projection (i.e., the mark resulting from deformation of the 2D brush tip image due to the stylus pose) may be used as a 2D stamp to create a mark on the virtual canvas in response to contact between the stylus and tablet. This may create a more natural mapping between tablet stylus poses and the resulting marks for artists accustomed to the behavior of pencils, felt tip pens, airbrushes, or another natural media, compared to those employed in previous digital painting applications. In some embodiments, in order to create a more natural mapping of tablet stylus poses to two-dimensional (2D) brush tip deformation, a graphics application or image editing application (e.g., a natural media painting application) may use a 3D texture projection model, such as a custom projection model or one based on the existing 3D graphics literature. The application may be configured to model a tablet stylus as if it were a virtual projector, and as if a 2D brush tip image were projected from the tip of the stylus onto a virtual canvas. The user interface includes a color palette whereby a user may manually load a brush with paint if and when desired, and a “fill” user interface element (shown as a radio button) whereby a user may enable or disable an auto fill option

U.S. 66/965,454B1 discloses a system and method for enabling graphic design by means of a computer system. Employing the invention involves the use of one or more forms capable of interacting with a computer system or network, each form comprising sheet material such as paper which has coded data printed on it and which allows it to interact with the computer system by use of a sensing device operated by a user. Specifically, the method includes the steps of printing on demand, on a surface, a form containing information relating to a graphic design activity, and at the same time as printing said information, printing on the surface coded data indicative of an identity of the form and of at least one reference point of the form, receiving, in a computer system, indicating data from a sensing device regarding the identity of the form and a position of the sensing device relative to the form, the sensing device, when placed in an operative position relative to the form, sensing the indicating data using at least some of the coded data, and identifying, in the computer system and from the indicating data, at least one parameter relating to the graphic design activity. The invention can be employed by a user to create and edit graphic images, either by means of a single computer, or by way of operation over a network. Accordingly, in a preferred form, the present invention provides methods and systems which use a paper and pen-based interface for a computer system. This provides many significant benefits over traditional computer systems. The advantage of paper is that it is widely used to display and record information. Further, printed information is easier to read than information displayed on a computer screen. Moreover, paper does not run on batteries, can be read in bright light, or robustly accepts coffee spills or the like and is portable and disposable. Furthermore, the system allows for hand-drawing and hand-writing to be captured which affords greater richness of expression than input via a computer keyboard and mouse. The marking nib of the pen is typically a ballpoint which produces a fixed-width line of a single fixed color. Nevertheless, the system can be used to draw and paint interactively using a variety of line styles, colors, and brushes. Not unlike a drawing or painting application in a conventional graphical user interface, the application presents a palette of available styles which can be used to draw or paint in what is sometimes referred to as “2½ dimensions”, i.e. in two dimensions with depth ordering of objects. The colors and styles used typically only become visible when the drawing or canvas page is reprinted, which generally takes place on demand

U.S. Pat. No. 8,854,342B2 discloses a graphics application for simulating natural media drawing and painting may model a tablet stylus as if it were a virtual airbrush tool that sprays paint on a virtual canvas (tablet). Graphics application may provide a painting or drawing tool that the user may manipulate, for example via tablet and/or stylus, to create or edit content in image.

U.S. Pat. No. 5,646,650A discloses an electronic paintbrush and electronic palette which allow a user to generate images on a CRT screen in a manner which imitates traditional painting with paints on a canvas. The electronic brush has fiber optic bristles which channel light from a CRT screen to optical detectors. A microprocessor in the brush utilizes pulses generated by the incidence of the raster scan on photodiodes to determine the position of the brush, the pressure of the brush against the CRT screen, and its angular orientation about the longitudinal axis of the brush. The microprocessor utilizes color information generated by a CCD array to determine the area of contact of the brush with color swatches on the palette, and the angle between the longitudinal axis of the brush and the normal vector to the CRT screen. In an alternate embodiment the palette is also displayed on the CRT screen. The image on the CRT may also be manipulated by user input through the keyboard by way of the processor.

### BRIEF SUMMARY OF THE INVENTION

In some embodiments, a system for virtual design includes a computing device. The computing device includes a processor, a memory, and a displaying device. The memory stores instructions executable by the processor. The instructions can include a first application. The displaying device is coupled to the processor. In some embodiments, the processor runs a first application. The first application is adapted to assist designers with the virtual tattoo design. The first application is integrated with the computing device and includes a plurality of design features from which the designers choose to modify a design into a final design.

In some embodiments, the first application includes features that assist the designers in designing a tattoo scheme for a costumer. The plurality of design features on the first application includes features to: save a design with one or more different formats to the computing device, save a design with one or more different formats to an external device coupled to the computing device, create a portfolio of designs based on two or more designs, create multiple designs based on one or more categories of designs, select one or more lines in a design for printing, create 3D designs based on two or more 2D designs, select a color pallet from a plurality of color pallets to color a design, select a brush style from a plurality of brush styles to brush a design, select line sizes from a plurality of line sizes for a design, select shades from a plurality of shades for shading a design, and send a design to an external device coupled to the computing device.

### BRIEF DESCRIPTION OF THE DRAWINGS

FIG. 1 illustrates a touch and virtual application for art, designs and tattoos, in accordance with some embodiments.

FIG. 2 illustrates a touch and virtual application for art, designs and tattoos, in accordance with some embodiments.

In accordance with common practice some features illustrated in the drawings cannot be drawn to scale. Accordingly, the dimensions of some features can be arbitrarily expanded or reduced for clarity. In addition, some of the drawings cannot depict all of the components of a given system, method or device. Finally, like reference numerals can be used to denote like features throughout the specification and figures.

### DETAILED DESCRIPTION

It will also be understood that, although the terms first, second, etc. are, in some instances, used herein to describe various elements, these elements should not be limited by these terms. These terms are only used to distinguish one element from another. For example, a first device could be termed a second device, and, similarly, a second device could be termed a first device, without departing from the scope of the various described embodiments. The first device and the second device are both devices, but they are not the same device, unless the context clearly indicates otherwise.

The terminology used in the description of the various described embodiments herein is for the purpose of describing particular embodiments only and is not intended to be limiting. As used in the description of the various described embodiments and the appended claims, the singular forms “a”, “an”, and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including”, “comprises”, and/or “comprising”, when used in this specification, specify the presence of stated features, integers, steps, operations, elements, and/or components, but do not preclude the presence or addition of one or more other features, integers, steps, operations, elements, components, and/or groups thereof.

As used herein, the term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

It should be appreciated that in the development of any actual embodiment (as in any development project), numerous decisions must be made to achieve the developers' specific goals (e.g., compliance with system and business-related constraints), and that these goals will vary from one embodiment to another. It will also be appreciated that such development efforts might be complex and time consuming but would nevertheless be a routine undertaking for those of ordinary skill in the art of image capture having the benefit of this disclosure.

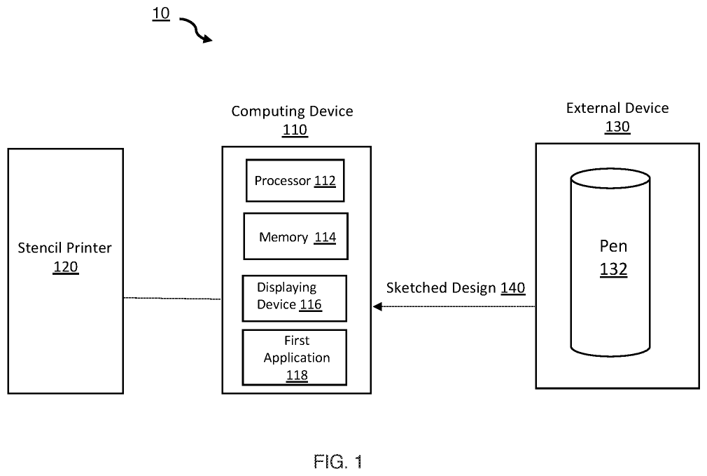

FIG. 1 illustrates a touch and virtual application for art, designs and tattoos, in accordance with some embodiments. In some embodiments, the system 10 includes a computing device 110. The computing device 110 includes a processor 112, a memory 114, and a displaying device 116. The memory stores instructions executable by the processor 112. The instructions can include a first application 118. The displaying device 116 is coupled to the processor 112. In some embodiments, the processor 112 runs a first application, e.g., the first application 118. The first application 118 is adapted to assist designers with the virtual tattoo design. The first application 118 is integrated with the computing device 110 and includes a plurality of design features from which the designers choose to modify a design into a final design.

In some embodiments, the first application 118 includes features that assist the designers in sketching a tattoo design for a costumer. The designers traditionally use papers or canvas to sketch tattoo designs. However, any modifications, such as deleting parts of the sketch, is a tedious, and even sometimes an almost-impossible, task, as the designer should be very careful not to alter any part of the sketch, other that undesired parts. To facilitate designers' job in sketching and modifying tattoo designs, the plurality of design features of the first application 118 includes features to: save a design with one or more different formats to the computing device, save a design with one or more different formats to an external device coupled to the computing device, create a portfolio of designs based on two or more designs, create multiple designs based on one or more categories of designs, select one or more lines in a design for printing, create 3D designs based on two or more 2D designs, select a color pallet from a plurality of color pallets to color a design, select a brush style from a plurality of brush styles to brush a design, select line sizes from a plurality of line sizes for a design, select shades from a plurality of shades for shading a design, and send a design to an external device coupled to the computing device 110. It should be noted that the designers can save all original designs on the computing device 110 or upload them to a suitable cloud platform.

In some embodiments, the system 10 includes an external device 130. The external device 130 is coupled to the computing device 110. The external device 130 includes a pen 132. The designer can use the pen to sketch a design on the external device 130. The external device 130 can be a tablet, a smartphone, a notebook, or any other suitable personal device. The designer can use the pen 132 to sketch a design on the external device 130 which can be subsequently sent to the computing device 110. On the computing device 110, the designer can use the first application 118 to modify the design and create the final design.

In some embodiments, the system 10 includes a stencil printer 120. The stencil printer 120 is in wireless communication with the computing device 110. The designer can send the modified design, e.g., the final design, by using the first application 118, to the stencil printer 120 for printing. The stencil printer 120 can receive the design from the computing device 110 and print a copy of the design. The printed design can be applied to a body of a user. The printed copy can be a removable ink copy. Alternatively, the printed copy can be a coloring ink.

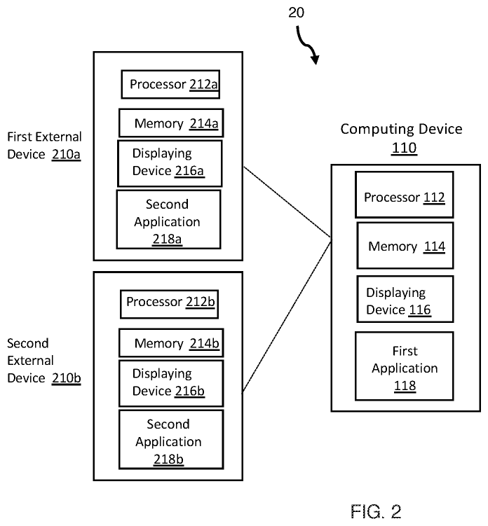

FIG. 2 illustrates a touch and virtual application for art, designs and tattoos, in accordance with some embodiments. In some embodiments, the system 20 includes a plurality of external user devices (e.g., a first external user device 210a, a second external user device 210b) in communication with the computing device 110. Each external user device includes a processor (e.g., a first processor 212a, a second processor 212b), a displaying device coupled to the processor (a first displaying device 216a, a second displaying device 216b), and a memory (a first memory 214a, a second memory 214b).

Each memory 214a and 214b stores instructions executable by a respective processor 212a and 212b. The instructions can include a second application (e.g., second applications 218a and 218b). Each displaying device 216a and 216b is coupled to a respective processor 212a and 212b. In some embodiments, each processor 212a and 212b runs a respective second application 218a and 218b. The second application 218a and 218b is integrated with respective external user device 210a and 210b. Each second application 218a and 218b is operable to receive a design from the designer and place the design on a photo of a user of the external user device 210a and 210b. Utilizing the second application enables the users to try their tattoo designs for placement on their body to their preference. Designers can send a draft design to the users, e.g., their clients, who use the second applications to see whether the clients approve the designs, or they need to be modified. Modifications, as previously disclosed, can be performed by using the first application 118 on the designer's device, e.g., the computing device 110.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best use the invention and various described embodiments with various modifications as are suited to the particular use contemplated.

What is claimed is:

1. A system for virtual tattoo design on a computing device, the system comprising:
a processor;
a displaying device coupled to the processor; and
a memory to store instructions executable by the processor;
wherein the processor is configured to:
run a first application adapted to assist designers with the virtual tattoo design, the first application is integrated with the computing device and comprising a plurality of design features from which the designers choose to modify a design into a final design.

2. The system of claim 1, wherein the plurality of design features comprising features to at least one of: save a design with one or more different formats to the computing device, save a design with one or more different formats to an external device coupled to the computing device, create a portfolio of designs based on two or more designs, create multiple designs based on one or more categories of designs, select one or more lines in a design for printing, create 3D designs based on two or more 2D designs, select a color pallet from a plurality of color pallets to color a design, select a brush style from a plurality of brush styles to brush a design, select line sizes from a plurality of line sizes for a design, select shades from a plurality of shades for shading a design, and send a design to an external device coupled to the computing device.

3. The system of claim 1, further comprising:
an external device coupled to the computing device, the external device comprising a pen, wherein the designer can use the pen to sketch a design on the external device.

4. The system of claim 1, further comprising:
a plurality of external user devices in communication with the computing device, each user external device comprising:
a processor;
a displaying device coupled to the processor; and
a memory to store instructions executable by the processor;
wherein the processor is configured to provide a second application, the second application is integrated with each external user device and operable to:
receive a design from the designer; and
place the design on a photo of a user of the external user device.

5. The system of claim 1, further comprising a stencil printer in communication with the computing device, the stencil printer operable to receive a design from the computing device and print a copy of the design, wherein the printed design can be applied to a body of a user.

6. The system of claim 5, wherein the printed copy is at least one of: a removable ink copy, and a coloring ink.

7. The system of claim 5, wherein the stencil printer is in wireless communication with the computing device.
